(19) United States
(12) Patent Application Publication
(10) Pub. No.: US 2025/0043336 A1
(43) Pub. Date: Feb. 6, 2025

(54) DETECTION OF 5-METHYLCYTOSINE

(71) Applicant: Pacific Biosciences of California, Inc., Menlo Park, CA (US)

(72) Inventors: Christopher T. SAUNDERS, Menlo Park, CA (US); Aaron WENGER, Menlo Park, CA (US)

(21) Appl. No.: 18/789,275

(22) Filed: Jul. 30, 2024

Related U.S. Application Data
(60) Provisional application No. 63/517,731, filed on Aug. 4, 2023.

(51) Int. Cl. C12Q 1/6827; G16B 20/20

(52) U.S. Cl. CPC C12Q 1/6827; G16B 20/20

(57) ABSTRACT

Methods, compositions, and systems are provided for the detection of 5-methylcytosine modifications in nucleic acid, particularly DNA, samples. A template or plurality of templates is sequenced using single molecule real time sequencing. Feature vectors are produced using specific features. In some aspects, a feature vector having a reduced feature set is provided and these feature vectors are input into a deep learning model.

### CROSS-REFERENCE TO RELATED APPLICATIONS

This application is based on, and claims the benefit of, U.S. Provisional Application No. 63/517,731, filed Aug. 4, 2023, which is incorporated herein by reference in its entirety.

### REFERENCE TO SEQUENCE LISTING

The instant application contains a Sequence Listing which has been submitted electronically in XML file format and is hereby incorporated by reference in its entirety. Said XML copy, created on Jul. 29, 2024, is named SL_49217_4014WO.xml and is 3,648 bytes in size.

### BACKGROUND OF THE INVENTION

It is becoming clearer that it is not only the genome sequence that is important in understanding human health but also the epigenome. In humans the typical epigenetic modification is the methylation of cytosines in the context of CpG sites. 5-methylcytosine (5mC), the most common form of DNA methylation, is involved in regulating many biological processes. In humans, most 5mCs occur at CpG sites, which are associated with cancer, embryonic development, and aging. Methylation patterns are known to be associated with human traits and diseases including syndromes such as Beckwith-Wiedemann syndrome, Prader-Willi syndrome and Angelman syndrome.

The importance of methylation in human phenotype has led to large-scale studies of human epigenomes in cell lines and tissues, such as the Roadmap Epigenomics Project and the ENCODE Consortium. Nearly all of these studies have used the approach of treating the DNA with bisulfite to convert non-methylated cytosines into uracils which are read by DNA sequencers and microarrays as thymines (Ts), while methylated cytosines are protected from conversion and read as Cs. The treated DNA is then measured with short read sequencers or microarrays and the difference between the uracil and the cytosine is used to call methylation status.

Single molecule real-time (SMRT) sequencing is also able to detect methylation, and it can do so with native DNA without treatment. This derives from the fundamental characteristics of SMRT sequencing which observes in real time a DNA polymerase synthesizing a DNA strand incorporating fluorescently labeled nucleotides. The fluorescent labels, for example the color of the of the pulses observed, indicate the identity of the base A, C, G, or T, and then the kinetics of the polymerase—for example how long it takes to incorporate a base and how long it goes between adjacent incorporations—is affected by both the context of the of the base and epigenetic modifications including methylation.

Statistical models, such as neural networks and deep learning, can be used to integrate the kinetics signal to detect methylation with high accuracy and throughput. There is a need for improved methods of applying these types of models for the accurate and high throughput detection of cytosine methylation modifications.

### BRIEF SUMMARY OF THE INVENTION

In some aspects, the invention provides methods for detecting 5-methylcytosine modifications in a nucleic acid template with a method that comprises: a) providing a nucleic acid template having a first strand and a complementary second strand, wherein the template is in a closed circular nucleic acid; b) subjecting the circular nucleic acid template to a real-time single molecule sequencing process that incorporates fluorescently labeled nucleotides into a nascent strand by a polymerase enzyme, and measuring emitted signals to obtain a set of traces comprising pulses; c) producing sequencing data by measuring features for each pulse in a trace of the real-time sequencing reaction, wherein said features comprise nucleotide identity, pulse width; and interpulse duration; d) creating, from the sequencing data, a set of feature vectors, each feature vector comprising two nucleotide positions comprising a known CpG site, at least 3 nucleotide positions upstream of the CpG site and at least three nucleotide positions downstream of the CpG site, each feature vector comprising the input features: (i) nucleotide identity for each nucleotide position in the feature vector, (ii) interpulse duration for each nucleotide position in the feature vector, and (iii) the average pulse width value for two or more nucleotides in the feature vector, the average pulse width value provided at only a single position in the feature vector; e) inputting the set of feature vectors into a model, the model trained using training feature vectors created from sequencing data obtained in the same manner and having the same input features recited above, wherein a subset of the training feature vectors represent nucleic acids known to have 5-methylcytosine modifications, and a subset of the training feature vectors represent nucleic acids known to be free of 5-methylcytosine modifications; and f) using the model to detect whether a cytosine or C in the known CpG site in each feature vector has 5-methyl-C modification.

In some embodiments, the average pulse width value is the average pulse width value for two consecutive nucleotides. In some embodiments, the feature vector comprises at least 5 nucleotide positions upstream and 5 nucleotide positions downstream of the known CpG site. In some embodiments, the feature vector comprises 16 nucleotide positions. In some embodiments, the feature vector has input features corresponding to the first strand. In some embodiments, the feature vector has input features corresponding to both the first strand and the complementary second strand. In some embodiments, a first feature vector with input features corresponding to the first strand and a second feature vector with input features corresponding to the second strand are input into the model to be processed by the model separately. In some embodiments, the processed features from the first feature vector and second feature vector are combined. In some embodiments, the processed features are combined using Bayesian inversion. In some embodiments, the input features comprise consensus values obtained by combining multiple sequencing reads.

In some embodiments, the model comprises a neural network model. In some embodiments, the neural network model comprises a convolutional neural network. In some embodiments, the model comprises a deep learning model. In some embodiments, the deep learning model comprises convolutional and pooling layers. In some embodiments, the deep learning model comprises a full connection layer. In some embodiments, the nucleic acid template is within a whole genome sequencing sample. In some embodiments, 5-methyl-C modifications are detected in a plurality of templates in the whole genome sequencing sample.

In some embodiments, the nucleic acid template comprises human DNA. In some embodiments, the circular nucleic acid template comprises a fragment from between 10,000 and 15,000 bases connected at both ends by hairpin structures. In some embodiments, the method is carried out on a sample comprising a plurality of nucleic acid templates.

### BRIEF DESCRIPTION OF THE DRAWINGS

FIG. 1A shows a polymerase enzyme as it incorporates a fluorescent nucleotide into a growing nascent strand representing the nucleic acid sequence of SEQ ID NO: 1.

FIG. 1B shows an exemplary trace and pulses for the nucleic acid sequence of SEQ ID NO: 2, illustrating how the determination of features from pulses in the trace can be performed.

FIG. 2 shows a distribution of number of passes for a library sequenced with HiFi single molecule real time (SMRT) sequencing.

FIG. 3 shows exemplary feature vectors of the invention for a forward strand.

FIG. 4 shows exemplary feature vectors of the invention for a forward strand and a reverse strand.

FIG. 5 shows a schematic of a deep learning model of using a single type of feature vector as input.

FIG. 6 shows a schematic of a deep learning model of using one feature vector for the first strand (SEQ ID NO: 3) and one feature vector for the complementary second strand as input.

FIG. 7 shows data illustrating accuracy obtained from the methods of the invention.

### DETAILED DESCRIPTION OF THE INVENTION

The present invention is generally directed to methods, compositions, and systems for detecting methylation modifications within nucleic acid sequences, and in particularly preferred aspects, 5-methylcytosine (5-methyl-C) nucleotides within templates through the use of single molecule nucleic acid sequencing.

The methods of the invention allow for detecting a 5-methyl-C modification in a nucleic acid template, for example a DNA template. The method of the invention includes carrying out real-time single molecule sequencing of the template. The sequencing process may use a closed circular nucleic acid which includes the template. The real-time single molecule sequencing process uses a single polymerase enzyme, which is optically monitored while it incorporates fluorescently labeled nucleotides into a nascent strand. Fluorescent signals from the incorporation events are measured and sequencing data is produced. These measurements provide traces having a series of pulses in which the pulses correspond to nucleotide incorporation events. Sequencing data is then created by measuring a set of features for each pulse in the traces. The features that are measured include nucleotide identity and nucleotide position, which are features corresponding to the nucleotide sequence of the nucleic acid. The features that are measured also include pulse width and interpulse duration, which are features known to correlate with the kinetics of polymerase mediated incorporation of the nucleotides.

Sequencing data obtained as described above is used for training a model, typically a deep learning model, to identify nucleotides that are 5-methyl-C modified. The same type of sequencing data is then obtained from nucleic acid samples to allow the trained model to detect 5-methyl-C modified bases in these nucleic acid samples.

To train the model and to have the model work on samples, a set of feature vectors is created from the sequencing data. Each feature vector is derived from a sequenced segment that has within it a known CpG site. In addition, the feature vectors have at least the following input features: (i) nucleotide identity for each nucleotide position in the feature vector, (ii) interpulse duration for each nucleotide position in the feature vector, and (iii) the average pulse width value for two or more nucleotides in the feature vector, wherein the average pulse width value provided at only a single position in the feature vector. In some embodiments, a first subset of the training feature vectors represent nucleic acids known to have 5-methylcytosine modifications, and a second subset of the training feature vectors represent nucleic acids known to be free of 5-methylcytosine modifications. In some embodiments, training the model includes using about 100,000 training feature vectors representing fully methylated nucleic acids (true positive) reads and 100,000 training feature vectors representing unmethylated nucleic acids (true negative) reads. In some embodiments, training the model includes using about less than 100,000 training feature vectors representing fully methylated nucleic acids reads and less than 100,000 training feature vectors representing unmethylated nucleic acids reads. In other embodiments, training the model includes using about more than 100,000 training feature vectors representing fully methylated nucleic acids reads and more than 100,000 training feature vectors representing unmethylated nucleic acids reads. In some embodiments, each read includes about 300 CpG sites.

In some aspects of the disclosure, provided herein is model training that uses approximately 100,000 true positive (fully methylated) and 100,000 true negative (unmethylated) HiFi reads from multiple SMRT Cells, which is described further herein. In some embodiments, each read has around 300 CpG sites, providing about 30 million true positive and 30 million true negative examples.

The inventors have found that this set of input features provides for accurate prediction of methylation within a template nucleic acid and provides a model with a reduced number of input features compared to a model that includes values for all nucleotide positions for all features. It is known in the art that reducing the number of features input into a neural network model can result in a more efficient, reliable, general, and stable predictive model.

In the art relating to models for predicting nucleotide modifications based on feature vectors created from single-molecule sequencing data, prior models used input data for all of the positions in a feature vector window for each property chosen to be input into the model. See, for example U.S. Pat. No. 11,091,794, which is incorporated herein by reference for all purposes. In the present invention, the inventors have unexpectedly found that, for the property of pulse width, an accurate model can be produced when only including a single average pulse width value at a single position in the feature vector. No value is input into the model that represents the pulse width for any specific nucleotide, but instead the input is a value that represents the average pulse width of two or more specified nucleotides within the feature vector, the nucleotides having known positions in the feature vector with respect to the CpG site.

To provide an accurate detection of 5-methyl-C modifications in a template, input values from at least three nucleotide positions upstream of the CpG site and three nucleotides downstream of the CpG site are typically included in the feature vector.

In one embodiment, two feature vectors are processed by the model, one feature vector for the first strand and one feature vector for the complementary second strand. The two feature vectors are each two-dimensional feature vectors that include 16 positions (columns). Each feature vector has a known CpG site with the C at position 7 and the G at position 8. The feature vector contains the identity of nucleotides for each position in the feature vector and contains the interpulse duration for each position in the feature vector. A single value, which is the average of pulse widths for positions 8 and 9 is put into position 8 of the feature vector. All other values for this property are set to 0. The two feature vectors are used to make two independent predictions of 5-methyl-C probability, each based on the evidence from one strand. Each prediction is produced by processing each feature vector through a subcomponent of the model designed to make predictions from individual strand evidence. The model can then combine the outputs of these processes to give a probability for 5-methyl-C modifications in the double stranded region of the nucleic acid representing by the feature vector.

In some embodiments, input features from only one of the strands are processed by the model. In some embodiments, a feature vector is input into the model that has the input features from both the first strand and the complementary second strand in the same feature vector.

The methods of the invention are directed toward detecting 5-methyl-C modifications in a nucleic acid template using a model. Detecting may include determining a probability that a 5-methyl-C modification is present at a cytosine within the template.

To determine the probabilities of methylation, relevant data must be obtained from a sample of interest that includes the template sequence. Typically, a sample has multiple templates. For example, in some cases, the methods of the invention are applied to human DNA samples covering the whole genome, having hundreds of thousands to millions of templates that can be analyzed for methylation status in a single experiment or a series of experiments.

Where single molecule real time (SMRT) sequencing is used, the DNA sample is typically provided as a library of fragments. In some cases, the library has a distribution of fragment lengths. Often, a library having a median length of around 10K to 15K base pairs is used. The fragment library can be converted into a library of closed circular nucleic acids by attaching hairpins to the ends of the fragments. These closed circular nucleic acids can then be used for circular consensus (CCS) sequencing.

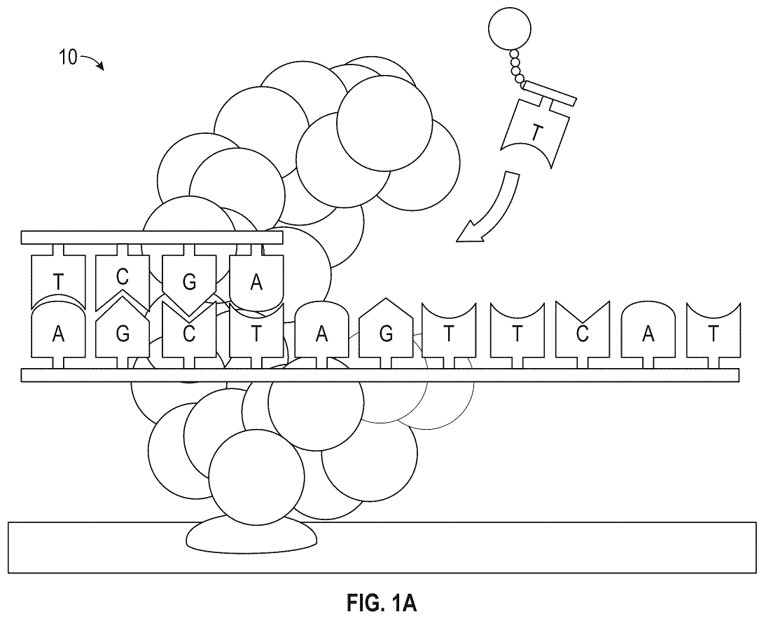

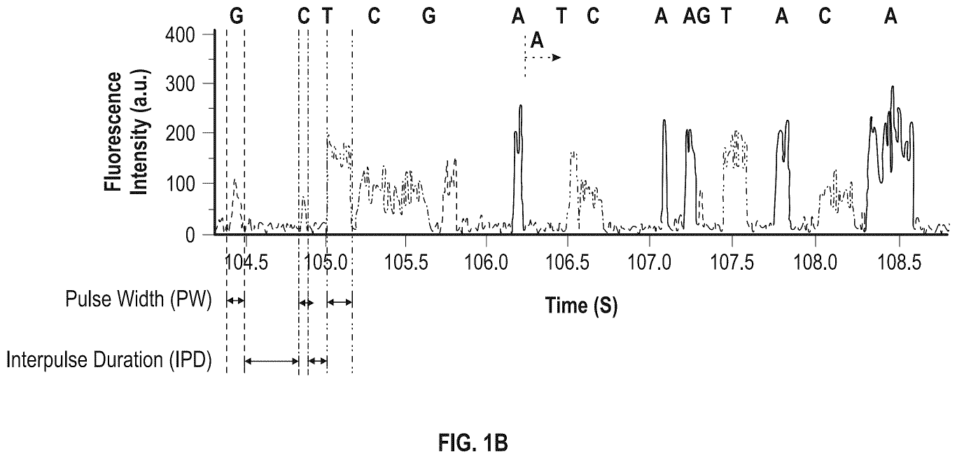

As recited herein and is well known in the art, SMRT sequencing uses fluorescent signals from labeled nucleotides as the nucleotides are incorporated into a growing nascent strand by a DNA polymerase enzyme. FIG. 1A illustrates how a polymerase enzyme 10 incorporates a fluorescently labeled nucleotide (T) into a growing nascent strand.

The nascent strand produced has the complementary sequence to the strand that the polymerase enzyme is copying, allowing the sequence of that portion of a template to be determined. The emitted fluorescent signals are recorded over time to produce a trace. The trace exhibits pulses, regions of the trace in which the fluorescent signal rises and then falls back to a baseline.

Sequencing data can be produced by analyzing the traces. Features of the pulses can be measured to produce this data. A number of pulse features can be determined. Some of the pulse features include pulse height (amplitude), pulse width (PW), and interpulse duration (IPD). Other pulse features such as the color (wavelength range) can be used. In some cases, the amplitude of the pulse is used, at least in part, to determine the nucleotide identity. FIG. 1B shows an example trace exhibiting pulses. Above the pulses is provided the nucleotide sequence determined from the pulse features. The figure also illustrates the determination of pulse width and interpulse duration from the trace. Note that a given pulse will generally have a single pulse width, but will generally have two interpulse durations, one measured between the pulse and the pulse preceding it and the other measured between the pulse and the one following it. Typically, when treating the data, a single pulse width is chosen by convention and provided for that pulse, for example the data used can be the IPD for the pulse and the pulse preceding it. Some features such as pulse width and interpulse duration are expressed in units of time. Any suitable unit of time can be used. In some cases, these features are expressed in units of number of frames. For a given sequencing system, the length of the frame is typically a fixed time, for example, 0.01 seconds.

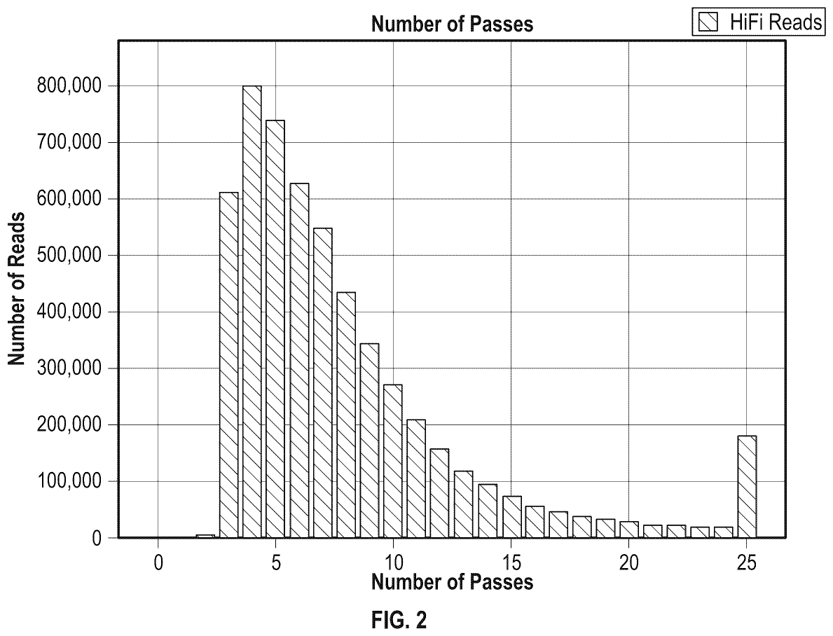

As recited above, circular consensus sequencing (CCS) or HiFi sequencing is used for the methods of the invention. CCS and HiFi sequencing produce multiple reads of the DNA fragments by sequencing around a circular DNA molecule such as a SMRTbell. CCS and HiFi can produce multiple reads for both the first strand (forward strand; fwd) and the complementary second strand (reverse strand; rev). As used herein, the terms “forward” and “reverse” designate the relative orientation and complementarity of the sequences and do not designate an absolute orientation of the sequences. The reads produced are often referred to as subreads. The number of subreads that are produced is referred to as the coverage. For example, if a sequencing run produces 3 full reads of the fwd strand and 2 full reads of the reverse strand, the coverage for the fwd strand is 3 and the coverage for the reverse strand is 2. In some cases, software is applied with criteria, e.g., a quality score, for determining whether a full subread has been produced. A quality score can be used, for example, to calculate the number of passes (coverage) for a subread or for individual nucleotides in a subread. Where a library of nucleotide fragments is used, there will be a distribution of the number of passes. FIG. 2 shows an example of a distribution of number of passes for a HiFi library.

The multiple passes from the CCS data is typically combined to produce a more accurate set of features corresponding to each nucleotide in the subread. For example, the values for the features from each of the subreads can be averaged for each nucleotide in the subread sequence. This can be done for all features including IPD and PW, separately for each strand or for the two strands together.

To determine whether a cytosine within a template is methylated, the features from the sequencing data are input into a model. As described herein, the model is typically a deep learning model. The model can be a convolutional neural network model.

For the methods of the invention, feature vectors are input into the model. The feature vectors are typically 2-dimensional feature vectors. For the instant invention, one axis of the two-dimensional feature vector is the nucleotide position. The feature vector has a known CpG site which takes two positions within the feature vector. The algorithm finds the CpG sites from the nucleotide sequence information from the sequencing data to produce the feature vectors. It is known that information in features upstream and downstream of the CpG site, such as IPD and PW, can be useful in determining whether the C in the CpG site is methylated. See, for example, U.S. Pat. No. 9,175,338 and Flusberg et al., Nature Methods, volume 7, page 461 (2010), which are incorporated herein by reference for all purposes. The feature vector typically includes input features for at least three nucleotides upstream and three nucleotides downstream of the CpG site. The number of nucleotide positions in the feature vector is typically between 8 nucleotides and 20 nucleotides, such as between 8 nucleotides and 20 nucleotides, between 8 nucleotides and 19 nucleotides, between 8 nucleotides and 18 nucleotides, between 8 nucleotides and 17 nucleotides, between 8 nucleotides and 16 nucleotides, between 8 nucleotides and 15 nucleotides, between 8 nucleotides and 14 nucleotides, between 8 nucleotides and 13 nucleotides, between 8 nucleotides and 12 nucleotides, between 8 nucleotides and 11 nucleotides, between 8 nucleotides and 10 nucleotides, between 9 nucleotides and 20 nucleotides, between 9 nucleotides and 20 nucleotides, between 10 nucleotides and 20 nucleotides, between 11 nucleotides and 20 nucleotides, between 12 nucleotides and 20 nucleotides, between 13 nucleotides and 20 nucleotides, between 14 nucleotides and 20 nucleotides, between 15 nucleotides and 20 nucleotides, between 16 nucleotides and 20 nucleotides, between 17 nucleotides and 20 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In some cases, the number of positions is between 12 and 18 nucleotides. In some cases, the number of positions is 16 nucleotides.

In some cases, the feature vector has features for both the forward strand and the reverse strand. In some cases, the feature vector has input features for only one of the forward or reverse strands.

The input features put into the feature vector of the invention include: 1) the identity of the nucleotide (A, G, C, or T) at each position, 2) interpulse duration values for each position, and 3) the average pulse width value for two or more nucleotides in the feature vector, the average pulse width value provided in a single position in the feature vector. All of the other positions in the row corresponding to average pulse width are typically set to zero.

The use of a single average pulse width in a single position within the feature vector deviates from the approaches that have been used in the past for applying neural network models to detecting modified bases. In past approaches, where a feature such as IPD or PW is used, a value is provided for each position in the window of the feature vector. Here, the inventors have found that by using a single value which represents an average pulse width an accurate prediction model can be created. This single value does not represent the actual pulse width for any specific nucleotide in the sequence. This approach allows for the input of fewer features into the model. Reducing the number of input features can result in a more reliable and stable model. The inventors have found that certain PW values are stronger predictors of methylation, and that the PW values can be combined (averaged) to a single value. This approach allows for reliance largely on IPD and only using a combination of the most indicative PW positions in the model.

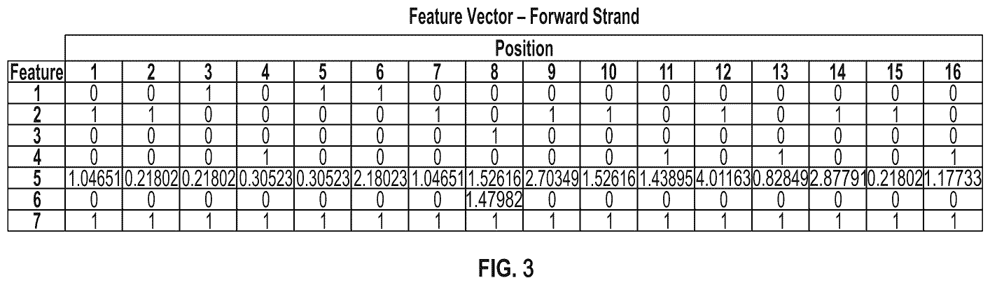

FIG. 3 provides an example of a feature vector of the instant invention with input features corresponding to a first or forward strand.

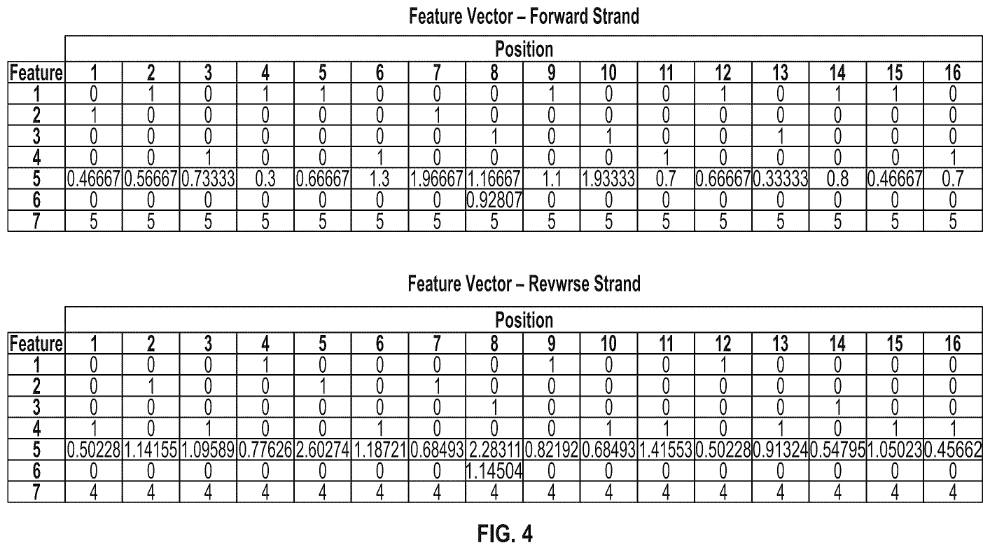

FIG. 4 provides an example of feature vectors of the instant invention. In this embodiment, two feature vectors, one for the first (fwd) strand, and one for the complementary second (rev) strand. The feature vectors have 16 positions (columns) corresponding to nucleotide position. Position 7 corresponds to a C of the CpG, and position 8 corresponds to position 8 of the CpG. There are 7 features (rows). Features 1-4 provide the nucleotide identity by 1-hot encoding, for example representing having 1, 2, 3, 4 represent A, C, G, T, and a 1 representing the nucleotide identity in that position, for example, a 1 in the row 2 indicating the nucleotide is a C. A nucleotide identity value is provided in each of the 16 positions of the feature vector.

Feature 5 is the interpulse duration, e.g., the duration between the pulse and the pulse preceding it. A value for interpulse duration is provided in each of the 16 positions of the feature vector.

Feature 6 has a value only in position 8. This value is an average of the pulse widths for the nucleotides at positions 8 and 9. These positions correspond to a G in the CpG site and the position one nucleotide downstream of the CpG site, which the inventors have determined are highly indicative of the presence methyl modification at a C of the CpG site.

Feature 7 represents the coverage, e.g. the number of passes that are combined to calculate a value at a particular nucleotide position. For FIG. 3 the coverage value is 1, in which case the features correspond to the values obtained in one trace. In FIG. 4, the coverage value for the forward strand is 5 and the coverage value for the reverse strand is 4. The values for the features in the table thus correspond to the combined value for the 5 traces for the forward strand and the combined value for 4 traces for the reverse strand. They combined value is typically the average or median value for the feature.

The model is trained by inputting feature vectors from training sets of sites in DNA known to be methylated and from DNA known to be unmethylated.

The trained model can be used to detect methylation in a sample having templates. As described above, SMRT sequencing is carried out on the sample and feature vectors typically including the same input features used to train the model are produced. The model determines a probability of whether the C in the CpG in the feature vector is methylated. This information can then be used to determine the probability that the cytosines within the one or more template nucleic acids are methylated. The probability determination (detection) can be determined for a single strand or can be determined taking into account both the forward (fwd) and reverse (rev) strands.

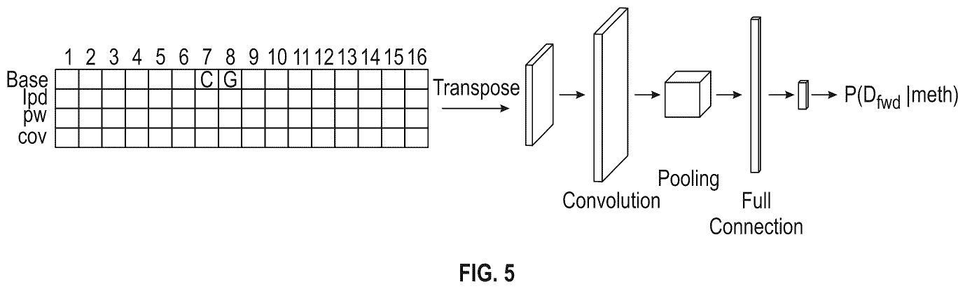

FIG. 5 shows an embodiment of a model for detecting methylation according to the invention. Feature vectors are created as described above and are input into a model. Here, the model has convolution, pooling, and full connection layers. The model output is a probability of methylation of the C in the CpG site. The feature vector can have information for one strand or can include information for the forward and reverse strands combined.

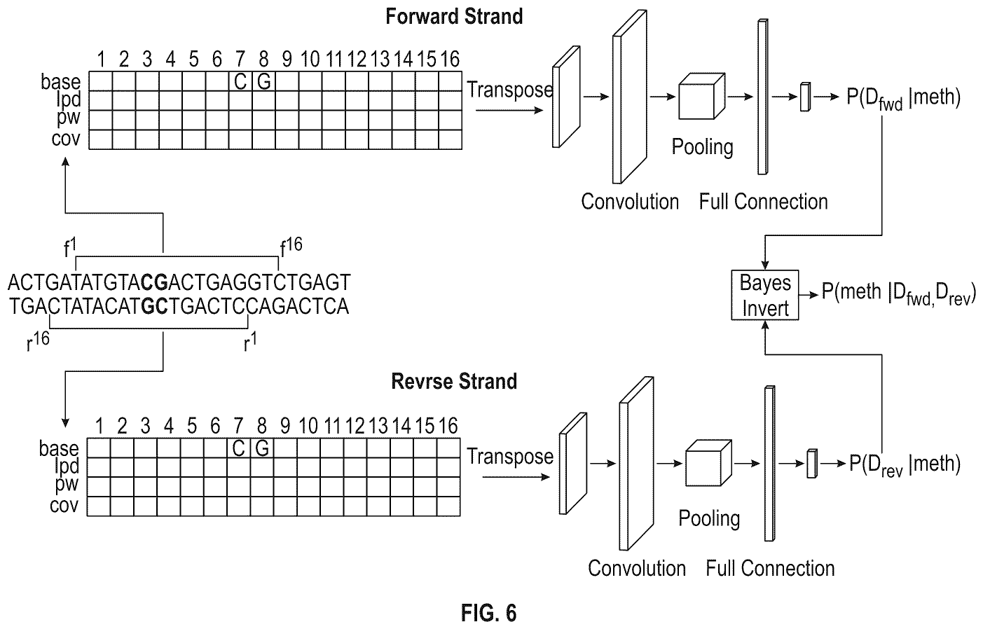

FIG. 6 shows an embodiment of a model for detecting methylation according to the invention. Feature vectors for each of the forward strand and reverse strand as described in FIG. 4 are created. These feature vectors are input into a deep learning model having convolutional, pooling, and full connection layers. From each of these, a probability of methylation for each strand is separately produced. These outputs are then combined to produce a probability that the double stranded DNA segment of the feature vector sequence is methylated. This figure shows one embodiment, which uses Bayesian inversion for combining the methylation probabilities for the two strands. Other processes for combining the probabilities for the forward and reverse strand can be used.

The ability to detect such modifications within nucleic acid sequences is useful for mapping such modifications in various types and/or sets of nucleic acid sequences, e.g., across a set of mRNA transcripts, across a chromosomal region of interest, or across an entire genome. The modifications so mapped can then be related to transcriptional activity, secondary structure of the nucleic acid, siRNA activity, mRNA translation dynamics, kinetics and/or affinities of DNA- and RNA-binding proteins, and other aspects of nucleic acid (e.g., DNA and/or RNA) metabolism.

Although certain embodiments of the invention are described in terms of detection of modified nucleotides or other modifications in a single-stranded DNA molecule (e.g., a single-stranded template DNA), various aspects of the invention are applicable to many different types of nucleic acids, including, e.g., single- and double-stranded nucleic acids that may comprise DNA (e.g., genomic DNA, mitochondrial DNA, viral DNA, etc.), RNA (e.g., mRNA, siRNA, microRNA, rRNA, tRNA, snRNA, ribozymes, etc.), RNA-DNA hybrids, PNA, LNA, morpholino, and other RNA and/or DNA hybrids, analogs, mimetics, and derivatives thereof, and combinations of any of the foregoing. Nucleic acids for use with the methods, compositions, and systems provided herein may consist entirely of native nucleotides, or may comprise non-natural bases/nucleotides (e.g., synthetic and/or engineered) that may be paired with native nucleotides or may be paired with the same or a different non-natural base/nucleotide. In certain preferred embodiments, the nucleic acid comprises a combination of single-stranded and double-stranded regions, e.g., such as the templates described in U.S. Ser. Nos. 12/383,855 and 12/413,258 and incorporated herein by reference in their entireties for all purposes.

Generally speaking, the methods of the invention involve monitoring a sequencing reaction to collect sequencing data, where the sequencing data is indicative of the progress of the reaction. Sequencing data includes data collected directly from the reaction to determine the nucleotide identity and position, as well as the results of various manipulations of that directly collected data, any or a combination of which can serve as a signal for the presence of a modification in the template nucleic acid. For example, certain types of sequencing data are collected in real time during the course of the reaction, such as metrics related to reaction kinetics, affinity, rate, processivity, signal characteristics, and the like. As used herein, “kinetics,” “kinetic signature,” “kinetic response,” “activity,” and “behavior” of an enzyme (or other reaction component, or the reaction as a whole) generally refer to reaction data related to the function/progress of the enzyme (or component or reaction) under investigation and are often used interchangeably herein.

Other types of data are generated from analysis of real time reaction data, including, e.g., accuracy, precision, conformance, etc. In some embodiments, data from a source other than the reaction being monitored is also used. For example, a sequence read generated during a nucleic acid sequencing reaction can be compared to sequence reads generated in replicate experiments, or to known or derived reference sequences from the same or a related biological source. Alternatively, or additionally, a portion of a template nucleic acid preparation can be amplified using unmodified nucleotides and subsequently sequenced to provide an experimental reference sequence to be compared to the sequence of the original template in the absence of amplification. Although certain specific embodiments of the use of particular types of sequencing data to detect certain kinds of modifications are described at length herein, it is to be understood that the methods, compositions, and systems are not limited to these specific embodiments.

In certain embodiments, redundant sequence information is generated and analyzed to detect one or more modifications in a template nucleic acid. Redundancy can be achieved in various ways, including carrying out multiple sequencing reactions using the same original template, e.g., in an array format, e.g., a ZMW array. In some embodiments, in which a lesion is unlikely to occur in all the copies of a given template, reaction data (e.g., sequence reads, kinetics, signal characteristics, signal context, and/or results from further statistical analyses) generated for the multiple reactions can be combined and subjected to statistical analysis to determine a consensus sequence for the template. In this way, the reaction data from a region in a first copy of the template can be supplemented and/or corrected with reaction data from the same region in a second copy of the template.

Modifications to DNA and RNA are well known. The methods of the invention are particularly suited for the measurement of methylation modifications, specifically 5-methylcytosine modifications. These and other modifications are known to those of ordinary skill in the art and are further described, e.g., in Narayan P, et al. (1987) Mol Cell Biol 7(4):1572-5; Horowitz S, et al. (1984) Proc Natl Acad Sci U.S.A. 81(18):5667-71; “RNA's Outfits: The nucleic acid has dozens of chemical costumes,” (2009) C&EN; 87(36):65-68; Kriaucionis, et al. (2009) Science 324 (5929): 929-30; and Tahiliani, et al. (2009) Science 324 (5929): 930-35; Matray, et al. (1999) Nature 399(6737):704-8; Ooi, et al. (2008) Cell 133: 1145-8; Petersson, et al. (2005) J Am Chem Soc. 127(5):1424-30; Johnson, et al. (2004) 32(6):1937-41; Kimoto, et al. (2007) Nucleic Acids Res. 35(16):5360-9; Ahle, et al. (2005) Nucleic Acids Res 33(10):3176; Krueger, et al., Curr Opinions in Chem Biology 2007, 11(6):588); Krueger, et al. (2009) Chemistry & Biology 16(3):242; McCullough, et al. (1999) Annual Rev of Biochem 68:255; Liu, et al. (2003) Science 302(5646):868-71; Limbach, et al. (1994) Nucl. Acids Res. 22(12):2183-2196; Wyatt, et al. (1953) Biochem. J. 55:774-782; Josse, et al. (1962) J. Biol. Chem. 237:1968-1976; Lariviere, et al. (2004) J. Biol. Chem. 279:34715-34720; and in International Application Publication No. WO/2009/037473, the disclosures of which are incorporated herein by reference in their entireties for all purposes.

In certain aspects, methods, compositions, and systems for detection of modifications in a template for single-molecule sequencing are provided, as well as determination of their location (i.e., “mapping”) within a nucleic acid molecule. In certain preferred embodiments, high-throughput, real-time, single-molecule, template-directed sequencing assays are used to detect the presence of such modified sites and to determine their location on the DNA template, e.g., by monitoring the progress and/or kinetics of a polymerase enzyme processing the template.

In certain aspects of the invention, single molecule real time sequencing systems are applied to the detection of modified nucleic acid templates through analysis of the sequence data including kinetic data derived from such systems. In particular, modifications in a template nucleic acid strand such as methylation alter the enzymatic activity of a nucleic acid polymerase in various ways, e.g., by increasing the time for a bound nucleotide to be incorporated and/or increasing the time between incorporation events. In certain embodiments, polymerase activity is detected using a single molecule nucleic acid sequencing technology. In certain embodiments, polymerase activity is detected using a nucleic acid sequencing technology that detects incorporation of nucleotides into a nascent strand in real time. In preferred embodiments, a single molecule nucleic acid sequencing technology is capable of real-time detection of nucleotide incorporation events. Such sequencing technologies are known in the art and include, e.g., the SMRT™ sequencing.

With regards to nucleic acid sequencing, the term “template” typically refers to a nucleic acid molecule from which sequencing data is obtained. A template may comprise, e.g., DNA, or analogs, or derivatives thereof, as described elsewhere herein. Further, a template may be single-stranded, double-stranded, or may comprise both single- and double-stranded regions. A modification in a double-stranded template may be in the strand complementary to the newly synthesized nascent strand, or may by in the strand identical to the newly synthesized strand, i.e., the strand that is displaced by the polymerase. A sample having the template nucleic acid can be obtained from any method for generating DNA samples. In some cases, the template nucleic acid is referred to as the target nucleic acid.

The nucleic acids used in the methods herein may be essentially any type of nucleic acid amendable to the methods presented herein. For example, a target nucleic acid may be DNA (e.g., genomic DNA, mtDNA, etc.), RNA (e.g., mRNA, siRNA, etc.), cDNA, peptide nucleic acid (PNA), amplified nucleic acid (e.g., via PCR, LCR, or whole genome amplification (WGA)), nucleic acid subjected to fragmentation and/or ligation modifications, whole genomic DNA or RNA, or derivatives thereof (e.g., chemically modified, labeled, recoded, protein-bound or otherwise altered). For example, a target nucleic acid may be bound to a protein involved in initiation of replication, e.g., Φ29 terminal protein p3 or adenovirus terminal protein, which are described in the art, e.g., in Blanco, et al. (1985) Proc. Natl. Acad. Sci. USA 82:6404-8; Pe5alva, et al. (1982) Proc. Natl. Acad. Sci. USA 79:5522-6; Inciarte, et al. (1980) J. Virol. 34:187-199; Harding, et al. (1980) Virology 104:323-338; Rekosh, et al. (1977) Cell 11:283-295; and Carusi, E. A. (1977) Virology 76:390-4, the disclosures of which are incorporated herein by reference in their entireties for all purposes. The target nucleic acid may be linear, circular (including templates for circular redundant sequencing (CRS)), single- or double-stranded, and/or double-stranded with single-stranded regions (e.g., stem- and loop-structures). For example, certain preferred template structures are provided in U.S. Ser. No. 12/413,258, filed Mar. 27, 2009.

The target nucleic acid may be purified or isolated from an environmental sample (e.g., ocean water, ice core, soil sample, etc.), a cultured sample (e.g., a primary cell culture or cell line), samples infected with a pathogen (e.g., a virus or bacterium), a tissue or biopsy sample, a forensic sample, a blood sample, or another sample from an organism, e.g., animal, plant, bacteria, fungus, virus, etc. Such samples may contain a variety of other components, such as proteins, lipids, and non-target nucleic acids. In certain embodiments, the target nucleic acid is a complete genomic sample from an organism. In other embodiments, the target nucleic acid is total RNA extracted from a biological sample or a cDNA library. As noted above, a target nucleic acid may be used directly in a template-directed sequencing reaction, or may be used to derive a population of nucleic acid templates suitable for use in such a reaction. For example, where whole genomic DNA is the target nucleic acid, it may be isolated from an organism, and fragmented to produce a population of template nucleic acids corresponding to the target nucleic acid. Further, target nucleic acid fragments or segments may be further subjected to size-selection (e.g., by chromatography, spin columns, or the like) to produce a pool of fragments within a desired size range (e.g., between about 500 and 5000 bp, or between about 700 and 2000 bp, or between about 500 and 20,000) or above a minimum size requirement, e.g., greater than about 250, 500, 1000, 2500, 5000, or 10,000 bp.

Isolation and/or purification of nucleic acids from samples is well known and routine in the art. Generally, nucleic acids can be extracted from a biological sample by a variety of techniques such as those described by Maniatis, et al., Molecular Cloning: A Laboratory Manual, Cold Spring Harbor, N.Y., pp. 280-281 (1982). A sample containing the target nucleic acid may be processed (e.g., homogenized or fractionated) in the presence of a detergent, surfactant, denaturant, reducing agent, and/or zwitterionic reagent by methods known in the art.

Detection of single molecules or molecular complexes in real time, e.g., during the course of an analytical reaction, generally involves direct or indirect disposal of the analytical reaction such that each molecule or molecular complex to be detected is individually resolvable. In this way, each analytical reaction can be monitored individually, even where multiple such reactions are immobilized on a single substrate. Individually resolvable configurations of analytical reactions can be accomplished through a number of mechanisms, and typically involve immobilization of at least one component of a reaction at a reaction site. Various methods of providing such individually resolvable configurations are known in the art, e.g., see European Patent No. 1105529 to Balasubramanian, et al.; and Published International Patent Application No. WO 2007/041394, the full disclosures of which are incorporated herein by reference in their entireties for all purposes. A reaction site on a substrate is generally a location on the substrate at which a single analytical reaction is performed and monitored, preferably in real time. A reaction site may be on a planar surface of the substrate, or may be in an aperture in the surface of the substrate, e.g., a well, nanohole, or other aperture. In preferred embodiments, such apertures are “nanoholes,” which are nanometer-scale holes or wells that provide structural confinement of analytic materials of interest within a nanometer-scale diameter, e.g., ˜1-300 nm. In some embodiments, such apertures comprise optical confinement characteristics, such as zero-mode waveguides, which are also nanometer-scale apertures and are further described elsewhere herein. Typically, the observation volume (i.e., the volume within which detection of the reaction takes place) of such an aperture is at the attoliter (10-18 L) to zeptoliter (10-21 L) scale, a volume suitable for detection and analysis of single molecules and single molecular complexes.

The immobilization of a component of an analytical reaction can be engineered in various ways. For example, an enzyme (e.g., polymerase, reverse transcriptase, kinase, etc.) may be attached to the substrate at a reaction site, e.g., within an optical confinement or other nanometer-scale aperture. In other embodiments, a substrate in an analytical reaction (for example, a nucleic acid template, e.g., DNA, RNA, or hybrids, analogs, or derivatives thereof may be attached to the substrate at a reaction site. Certain embodiments of template immobilization are provided, e.g., in U.S. patent application Ser. No. 12/562,690 and incorporated herein by reference in its entirety for all purposes. One skilled in the art will appreciate that there are many ways of immobilizing nucleic acids and proteins into an optical confinement, whether covalently or non-covalently, via a linker moiety, or tethering them to an immobilized moiety. These methods are well known in the field of solid phase synthesis and micro-arrays (Beier et al., Nucleic Acids Res. 27:1970-1-977 (1999)). Non-limiting exemplary binding moieties for attaching either nucleic acids or polymerases to a solid support include streptavidin or avidin/biotin linkages, carbamate linkages, ester linkages, amide, thiolester, (N)-functionalized thiourea, functionalized maleimide, amino, disulfide, amide, hydrazone linkages, among others. Antibodies that specifically bind to one or more reaction components can also be employed as the binding moieties. In addition, a silyl moiety can be attached to a nucleic acid directly to a substrate such as glass using methods known in the art.

In some embodiments, a nucleic acid template is immobilized onto a reaction site (e.g., within an optical confinement) by attaching a primer comprising a complementary region at the reaction site that is capable of hybridizing with the template, thereby immobilizing it in a position suitable for monitoring. In certain embodiments, an enzyme complex is assembled in an optical confinement, e.g., by first immobilizing an enzyme component. In other embodiments, an enzyme complex is assembled in solution prior to immobilization.

In some embodiments, a substrate comprising an array of reaction sites is used to monitor multiple sequencing reactions, each taking place at a single one of the reaction sites. Various means of loading multiple biological reactions onto an arrayed substrate are known to those of ordinary skill in the art and are described further in U.S. Pat. Nos. 8,906,831, 8,658,364, 10,300,452, 10,731,211, 10,814,299, 11,332,787 which are incorporated herein by reference in their entirety for all purposes.

In preferred aspects, the methods, compositions, and systems provided herein utilize optical confinements to facilitate single molecule resolution of analytical reactions. In preferred embodiments, such optical confinements are configured to provide tight optical confinement so only a small volume of the reaction mixture is observable. Some such optical confinements and methods of manufacture and use thereof are described at length in, e.g., U.S. Patent. Nos. 7,302,146. 7,476,503, 7,313,308, 7,315,019, 7,170,050, 6,917,726, 7,013,054, 7,181,122, and 7,292,742; U.S. Patent Publication Nos. 2008/0128627, 2008/0152281, and 2008/01552280; and U.S. Ser. Nos. 11/981,740 and 12/560,308, all of which are incorporated herein by reference in their entireties for all purposes.

In certain preferred embodiments of the invention, single-molecule real-time sequencing systems already developed are applied to the detection of modified nucleic acid templates through analysis of the sequence and kinetic data derived from such systems. As described below, methylated cytosine and other modifications in a template nucleic acid will alter the enzymatic activity of a polymerase processing the template nucleic acid. In certain embodiments, polymerase kinetics in addition to sequence read data are detected using a single molecule nucleic acid sequencing technology, e.g., the SMRT sequencing technology developed by Pacific Biosciences (Eid, J. et al. (2009) Science 2009, 323, 133, the disclosure of which is incorporated herein by reference in its entirety for all purposes). This technique is capable of long sequencing reads and provides high-throughput methylation profiling even in highly repetitive genomic regions, facilitating de novo sequencing of modifications such as methylated bases. SMRT sequencing systems typically utilize state-of-the-art single-molecule detection instruments, production-line nanofabrication chip manufacturing, organic chemistry, protein mutagenesis, selection and production facilities, and software and data analysis infrastructures.

Certain preferred methods of the invention employ real-time sequencing of single DNA molecules (Eid, et al., supra), with intrinsic sequencing rates of several bases per second and average read lengths in the kilobase range. In such sequencing, sequential base additions catalyzed by DNA polymerase into the growing complementary nucleic acid strand are detected with fluorescently labeled nucleotides. The kinetics of base additions and polymerase translocation are sensitive to the structure of the DNA double-helix, which is impacted by the presence of base modifications, e.g., 5-MeC, 5-hmC, base J, etc., and other perturbations (secondary structure, bound agents, etc.) in the template. By monitoring the activity of DNA polymerase during sequencing, sequence read information and base modifications can be simultaneously detected. Long, continuous sequence reads that are readily achievable using SMRT sequencing facilitate modification (e.g., methylation) profiling in low complexity regions that are inaccessible to some technologies, such as certain short-read sequencing technologies. Carried out in a highly parallel manner, methylomes can be sequenced directly, with single base-pair resolution and high throughput.

For the methods of the invention it is particularly useful to incorporate HiFi reads. These are single molecule consensus reads that can be produced by sequencing a closed circular template (e.g., SMRTbell) that includes a sequence of interest. The SMRTbell has a double stranded region connected on either side with hairpins. SMRT sequencing can proceed around these molecules, sequencing the same regions multiple times. The incorporation of a double-stranded nucleic acid fragment into a closed circular single-stranded template (e.g., as described in U.S. Patent Publication No. 2009/0298075) also allows for combination and comparison of the polymerase kinetics on the forward and reverse strand. Since the forward and reverse strands are reverse complements of each other, one must construct the expectation of the ratios of the parameters of interest (e.g., pulse width, IPD, sequence context, etc.) from an entirely unmodified sample, e.g., using amplification to produce amplicons that do not comprise the modification(s).

These types of sequencing reads can be both long and accurate. This type of sequencing can produce read lengths on the order of 15,000 to 25,000 bases with read level accuracies of 99.9% or higher for variant calling. This combination of read length and accuracy provides the most comprehensive characterization of human genomes in the area of variant calling. This single molecule consensus approach also allows for the accurate measure of kinetic features such as interpulse duration and pulse width by combining the information from multiple passes on the same region of DNA, both forward and reverse strands. Each of those observations are independent. The observations happen consecutively in time and each of those observations provides independent base calls which can be combined to create consensus and to generate an extremely accurate read. Each observation also measures kinetics independently, and while the signature of an epigenetic modification may be subtle in a single pass, observations over multiple passes of the molecule can be combined to generate more accurate methylation calls.

As described above, sequencing data includes data that is indicative of the progress of a reaction and can serve as a signal for the presence of a modification in the template nucleic acid. Sequencing data in single molecule sequencing reaction reactions using fluorescently labeled bases is generally centered around characterization of detected fluorescence pulses, a series of successive pulses (“trace” or one or more portions thereof), and other downstream statistical analyses of the pulse and trace data. Fluorescence pulses can be characterized not only by their spectrum, but also by other metrics including their duration, shape, intensity, and by the interval between successive pulses (see, e.g., Eid, et al., supra; and U.S. Patent Publication No. 2009/0024331, incorporated herein by reference in its entirety for all purposes).

These metrics provide valuable information about the processing of a template, e.g., the kinetics of nucleotide incorporation and DNA polymerase processivity and other aspects of the reaction. Further, the context in which a pulse is detected (e.g. the nucleotide sequence context) can contribute to the identification of the pulse. For example, the presence of a methyl modification alters not only the processing of the template at the site of the modification, but also the processing of the template upstream and/or downstream of the modification. For example, the presence of methylated nucleotides in a template nucleic acid has been shown to change the width of a pulse (PW) and/or the interpulse duration (IPD), either at the position of the modified base or at one or more positions proximal to it. The inventors have found that for the detection of 5-methyl-C, the IPDs are changed for positions relatively far from the site of modification, but that the PW is only changed within a couple of nucleotides of the site of modification.

In yet further embodiments, sequencing data is generated by analysis of the pulse and trace data to determine error metrics for the reaction. Such error metrics include not only raw error rate, but also more specific error metrics, e.g., identification of pulses that did not correspond to an incorporation event, incorporations that were not accompanied by a detected pulse, incorrect incorporation events, and the like. Any of these error metrics, or combinations thereof, can serve as a signal indicative of the presence of one or more modifications in the template nucleic acid. In some embodiments, such analysis involves comparison to a reference sequence and/or comparison to replicate sequence information from the same or an identical template, e.g., using a standard or modified multiple sequence alignment.

Although described herein primarily with regards to fluorescently labeled nucleotides, other types of detectable labels and labeling systems can also be used with the methods, compositions, and systems described herein including, e.g., quantum dots, surface enhanced Raman scattering particles, scattering metallic nanoparticles, FRET systems, intrinsic fluorescence, non-fluorescent chromophores, and the like. Such labels are generally known in the art and are further described in U.S. Pat. Nos. 6,399,335, 5,866,366, 7,476,503, and 4,981,977; U.S. Patent Pub. No. 2003/0124576; U.S. Ser. No. 61/164,567; WO 01/16375; Mujumdar, et al Bioconjugate Chem. 4(2):105-111, 1993; Ernst, et al, Cytometry 10:3-10, 1989; Mujumdar, et al, Cytometry 10:1119, 1989; Southwick, et al, Cytometry 11:418-430, 1990; Hung, et al, Anal. Biochem. 243(1):15-27, 1996; Nucleic Acids Res. 20(11):2803-2812, 1992; and Mujumdar, et al, Bioconjugate Chem. 7:356-362, 1996; Intrinsic Fluorescence of Proteins, vol. 6, publisher: Springer US, ©2001; Kronman, M. J. and Holmes, L. G. (2008) Photochem and Photobio 14(2): 113-134; Yanushevich, Y. G., et al. (2003) Russian J. Bioorganic Chem 29(4) 325-329; and Ray, K., et al. (2008) J. Phys. Chem. C 112(46): 17957-17963, all of which are incorporated herein by reference in their entireties for all purposes. Many such labeling groups are commercially available, e.g., from the Amersham Biosciences division of GE Healthcare, and Molecular Probes/Invitrogen Inc. (Carlsbad, CA)., and are described in ‘The Handbook—A Guide to Fluorescent Probes and Labeling Technologies, Tenth Edition’ (2005) (available from Invitrogen, Inc./Molecular Probes and incorporated herein in its entirety for all purposes). Further, a combination of the labeling strategies described herein and known in the art for labeling reaction components can be used.

Various different polymerases may be used in template-directed sequence reactions, e.g., those described at length, e.g., in U.S. Pat. No. 7,476,503, the disclosure of which is incorporated herein by reference in its entirety for all purposes. In brief, the polymerase enzymes suitable for the present invention can be any nucleic acid polymerases that are capable of catalyzing template-directed polymerization with reasonable synthesis fidelity. The polymerases can be DNA polymerases or RNA polymerases (including, e.g., reverse transcriptases), DNA-dependent or RNA-dependent polymerases, thermostable polymerases or thermally degradable polymerases, and wildtype or modified polymerases. In some embodiments, the polymerases exhibit enhanced efficiency as compared to the wildtype enzymes for incorporating unconventional or modified nucleotides, e.g., nucleotides linked with fluorophores. In certain preferred embodiments, the methods are carried out with polymerases exhibiting a high degree of processivity, i.e., the ability to synthesize long stretches (e.g., over about 10 kilobases) of nucleic acid by maintaining a stable nucleic acid/enzyme complex. In certain preferred embodiments, sequencing is performed with polymerases capable of rolling circle replication. A preferred rolling circle polymerase exhibits strand-displacement activity, and as such, a single circular template can be sequenced repeatedly to produce a sequence read comprising multiple copies of the complement of the template strand by displacing the nascent strand ahead of the translocating polymerase. Since the methods of the invention can increase processivity of the polymerase by removing lesions that block continued polymerization, they are particularly useful for applications in which a long nascent strand is desired, e.g. as in the case of rolling-circle replication. Non-limiting examples of rolling circle polymerases suitable for the present invention include but are not limited to T5 DNA polymerase, T4 DNA polymerase holoenzyme, phage M2 DNA polymerase, phage PRD1 DNA polymerase, Klenow fragment of DNA polymerase, and certain polymerases that are modified or unmodified and chosen or derived from the phages Φ29 (Phi29), PRD1, Cp-1, Cp-5, Cp-7, Φ15, Φ1, Φ21, Φ25, BS 32 L17, PZE, PZA, Nf, M2Y (or M2), PR4, PR5, PR722, B103, SF5, GA-1, and related members of the Podoviridae family. In certain preferred embodiments, the polymerase is a modified Phi29 DNA polymerase, e.g., as described in U.S. Patent Publication No. 20080108082, incorporated herein by reference in its entirety for all purposes. Additional polymerases are provided, e.g., in U.S. Ser. No. 11/645,125, filed Dec. 21, 2006; Ser. No. 11/645,135, filed Dec. 21, 2006; Ser. No. 12/384,112, filed Mar. 30, 2009; and 61/094,843, filed Sep. 5, 2008; as well as in U.S. Patent Publication No. 20070196846, the disclosures of which are incorporated herein by reference in their entireties for all purposes.

Treatment and analysis of the data generated by the methods described herein includes methods using software and/or statistical algorithms that perform various data conversions, e.g., conversion of signal emissions into basecalls, conversion of basecalls into consensus sequences for a nucleic acid template, and conversion of various aspects of the basecalls and/or consensus sequence to derive a reliability metric for the resulting values. Such software, statistical algorithms, and use thereof are described in detail, e.g., in U.S. Pat. Nos. 8,370,079 and 8,703,422, the disclosures of which are incorporated herein by reference in their entireties for all purposes. Specific methods for discerning altered nucleotides in a template nucleic acid are provided in U.S. Pat. Nos. 9,175,341, 8,940,507, 9,238,836, and 9,116,118, which are incorporated herein by reference in its entirety for all purposes.

Models employed in the invention include deep learning and machine learning models. In some embodiments, deep learning algorithms (e.g., convolutional neural networks (CNN)) are used, for example for distinguishing the methylated CpGs from unmethylated CpGs. Other algorithms include, but are not limited to, linear regression, logistic regression, deep recurrent neural network (e.g., long short-term memory, LSTM), Bayes classifier, hidden Markov model (HMM), linear discriminant analysis (LDA), k-means clustering, density-based spatial clustering of applications with noise (DBSCAN), random forest algorithm, and support vector machine (SVM), etc.

In some cases, machine learning algorithms are employed. Non-limiting examples of machine learning algorithms include supervised algorithms (such as algorithms where the features/classifications in the data set are annotated) using linear regression, logistic regression, decision trees, classification and regression trees, naïve Bayes, nearest neighbor clustering; unsupervised algorithms (such as algorithms where no features/classification in the data set are annotated) using a priori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

In certain aspects, the invention provides methods for detecting changes in the kinetics and other reaction data for real-time DNA sequencing. As discussed at length above, detection of a change in such sequencing kinetics applications can be indicative the presence of modifications such as 5-methyl-C modifications in the template, the presence of an agent bound to the template, and the like. It is appreciated that the kinetic activity of single molecules does not follow the regular and simple picture implied by traditional chemical kinetics, a view dominated by single-rate exponentials and the smooth results of ensemble averaging. In a large multi-dimensional molecular system, such as the polymerase-DNA complex, there are processes taking place on many different time scales, and the resultant kinetic picture can be quite complex at the molecular level. (See, e.g., Herbert, et al. (2008) Ann Rev Biochem 77:149.) General information on algorithms for use in sequence analysis can be found, e.g., in Braun, et al. (1998) Statist Sci 13:142; and Durbin, et al. (1998) Biological sequence analysis: Probabilistic models of proteins and nucleic acids, Cambridge University Press: Cambridge, UK.

As is known in the art, the models used, including neural network and deep learning models typically need to be trained. The models in the instant invention can be trained on training data that is produced using one set of nucleic acid that is fully methylated and one set of nucleic acids that is fully unmethylated. An approach to produce the sequences for training is to start with native human DNA, for example, the Human DNA sample HG002. In the native DNA some of the sites are methylated and some are not. Whole genome amplification is performed using a PCR amplification which produces an effectively fully unmethylated DNA library. A fully C-methylated sample is created by treating some of the fully unmethylated DNA with a CpG methyltransferase enzyme that efficiently adds methylation to any CpG site, producing fully C-methylated sequences. These two sets of DNA are used to provide truth sets to train the model. As is typical in such training, some regions of the genome were held out to allow evaluation of precision and recall. Those of skill in the art will understand how to train a model such as a deep learning model.

The invention also provides systems that are used in conjunction with the compositions and methods of the invention to provide for real-time single-molecule detection of analytical reactions. In particular, such systems typically include the reagent systems described herein, in conjunction with an analytical system, e.g., for detecting data from those reagent systems. In certain preferred embodiments, analytical reactions are monitored using an optical system capable of detecting and/or monitoring interactions between reactants at the single-molecule level. For example, such an optical system can achieve these functions by first generating and transmitting an incident wavelength to the reactants, followed by collecting and analyzing the optical signals from the reactants.

Such systems may employ an optical train that directs signals from the reactions to a detector, and in certain embodiments in which a plurality of reactions is disposed on a solid surface, such systems typically direct signals from the solid surface (e.g., array of confinements) onto different locations of an array-based detector to simultaneously detect multiple different optical signals from each of multiple different reactions. In particular, the optical trains may include optical gratings or wedge prisms to simultaneously direct and separate signals having differing spectral characteristics from each confinement in an array to different locations on an array based detector, e.g., a CCD, and may also comprise additional optical transmission elements and optical reflection elements. In some cases, systems include integrated chips having fluidic, optical, and electronic components. See, for example, U.S. Pat. Nos. 8,465,699, 9,291,569, 8,467,061, 9,372,308, 9,223,084, 9,624,540, and 9,606,068, which are incorporated herein by reference for all purposes.

An optical system applicable for use with the present invention typically comprises at least an excitation source and a photon detector. The excitation source generates and transmits incident light used to optically excite the reactants in the reaction. Depending on the intended application, the source of the incident light can be a laser, laser diode, a light-emitting diode (LED), a ultra-violet light bulb, and/or a white light source. Further, the excitation light may be evanescent light, e.g., as in total internal reflection microscopy, certain types of waveguides that carry light to a reaction site (see, e.g., U.S. Application Pub. Nos. 2008/0128627, 2008/0152281, and 2008/01552280), or zero mode waveguides, described below. Where desired, more than one source can be employed simultaneously. The use of multiple sources is particularly desirable in applications that employ multiple different reagent compounds having differing excitation spectra, consequently allowing detection of more than one fluorescent signal to track the interactions of more than one or one type of molecules simultaneously (e.g., multiple types of differentially labeled reaction components). A wide variety of photon detectors or detector arrays are available in the art. Representative detectors include but are not limited to an optical reader, a high-efficiency photon detection system, a photodiode (e.g., avalanche photo diodes (APD)), a camera, a charge-coupled device (CCD), an electron-multiplying charge-coupled device (EMCCD), an intensified charge coupled device (ICCD), and a confocal microscope equipped with any of the foregoing detectors. For example, in some embodiments an optical train includes a fluorescence microscope capable of resolving fluorescent signals from individual sequencing complexes. Where desired, the subject arrays of optical confinements contain various alignment aides or keys to facilitate a proper spatial placement of the optical confinement and the excitation sources, the photon detectors, or the optical train as described below.

In one embodiment, a reaction site (e.g., optical confinement) containing a reaction of interest is operatively coupled to a photon detector. The reaction site and the respective detector can be spatially aligned (e.g., 1:1 mapping) to permit an efficient collection of optical signals from the reactants. In certain preferred embodiments, a reaction substrate is disposed upon a translation stage, which is typically coupled to appropriate robotics to provide lateral translation of the substrate in two dimensions over a fixed optical train. Alternative embodiments could couple the translation system to the optical train to move that aspect of the system relative to the substrate. For example, a translation stage provides a means of removing a reaction substrate (or a portion thereof) out of the path of illumination to create a non-illuminated period for the reaction substrate (or a portion thereof), and returning the substrate at a later time to initiate a subsequent illuminated period. An exemplary embodiment is provided in U.S. Patent Pub. No. 2007/0161017, filed Dec. 1, 2006.

In particularly preferred aspects, such systems include arrays of reaction regions, e.g., zero mode waveguide arrays, that are illuminated by the system, in order to detect signals (e.g., fluorescent signals) therefrom, that are in conjunction with analytical reactions being carried out within each reaction region. Each individual reaction region can be operatively coupled to a respective microlens or a nanolens, preferably spatially aligned to optimize the signal collection efficiency. Alternatively, a combination of an objective lens, a spectral filter set or prism for resolving signals of different wavelengths, and an imaging lens can be used in an optical train, to direct optical signals from each confinement to an array detector, e.g., a CCD, and concurrently separate signals from each different confinement into multiple constituent signal elements, e.g., different wavelength spectra, that correspond to different reaction events occurring within each confinement. In preferred embodiments, the setup further comprises means to control illumination of each confinement, and such means may be a feature of the optical system or may be found elsewhere is the system, e.g., as a mask positioned over an array of confinements. Detailed descriptions of such optical systems are provided, e.g., in U.S. Patent Pub. No. 2006/0063264 which is incorporated herein by reference in its entirety for all purposes.

The systems of the invention also typically include information processors or computers operably coupled to the detection portions of the systems, to store the signal data obtained from the detector(s) on a computer readable medium, e.g., hard disk, CD, DVD or other optical medium, flash memory device, or the like. For purposes of this aspect of the invention, such operable connection provides for the electronic transfer of data from the detection system to the processor for subsequent analysis and conversion. Operable connections may be accomplished through any of a variety of well known computer networking or connecting methods, e.g., Firewire®, USB connections, wireless connections, WAN or LAN connections, or other connections that preferably include high data transfer rates. The computers also typically include software that analyzes the raw signal data, identifies signal pulses that are likely associated with incorporation events, and identifies bases incorporated during the sequencing reaction, in order to convert or transform the raw signal data into user interpretable sequence data (see, e.g., Published U.S. Patent Pub. No. 2009/0024331, the full disclosure of which is incorporated herein by reference in its entirety for all purposes). Exemplary systems are described in detail in, e.g., U.S. Pat. Nos. 8,465,699, 9,291,569, 8,467,061, 9,372,308, 9,223,084, 9,624,540, and 9,606,068, which are incorporated herein by reference for all purposes.

The invention provides data processing systems for transforming raw data generated in an analytical reaction into analytical data that provides a measure of one or more aspects of the reaction under investigation, e.g., transforming signals from a sequencing-by-synthesis reaction into nucleic acid sequence read data, which can then be transformed into consensus sequence data. In certain embodiments, the data processing systems include machines for generating nucleic acid sequence read data by polymerase-mediated processing of a template nucleic acid molecule (e.g., DNA or RNA). A nucleic acid sequence read generated is representative of the nucleic acid sequence of the nascent polynucleotide synthesized by a polymerase translocating along a nucleic acid template, but may not be identical to the actual sequence of the nascent polynucleotide molecule. For example, it may contain a deletion or a different nucleotide at a given position as compared to the actual sequence of the polynucleotide, e.g., when a nucleotide incorporation is missed or incorrectly determined, respectively. As such, it is beneficial to generate redundant nucleic acid sequence read data, and to transform the redundant nucleic acid sequence read data into consensus nucleic acid sequence data that is generally more representative of the actual sequence of the polynucleotide molecule than nucleic acid sequence read data from a single read of the nucleic acid molecule. Redundant nucleic acid sequence read data comprises multiple reads, each of which includes at least a portion of nucleic acid sequence read that overlaps with at least a portion of at least one other of the multiple nucleic acid sequence reads. An elegant way to produce redundant sequencing data is to use HiFi sequencing, which uses a circular construct formed by adding hairpins to the end of a double stranded nucleic acid. The SMRT sequencing proceeds around the circular template multiple times, providing multiple reads of both the forward strand and the reverse strand. The multiple reads can be combined to improve the accuracy of the nucleotide sequence information and the kinetic information (e.g., nucleotide identity, IPD and PW).

The data processing systems can include software and algorithm implementations provided herein, e.g., those configured to transform redundant sequence read data into consensus sequence data, which, as noted above, is generally more representative of the actual sequence of the nascent polynucleotide molecule and of the polymerase kinetics than nucleic acid sequence read data from a single read of a single nucleic acid molecule.

The software and algorithm implementations provided herein are preferably machine-implemented methods, e.g., carried out on a machine comprising computer-readable medium configured to carry out various aspects of the methods herein. For example, the computer-readable medium can have at least one or more of the following: a) a user interface; b) memory for storing raw analytical reaction data; c) memory storing software-implemented instructions for carrying out the algorithms for transforming the raw analytical reaction data into transformed data that characterizes one or more aspects of the reaction (e.g., rate, consensus sequence data, etc.); d) a processor for executing the instructions; e) software for recording the results of the transformation into memory; and f) memory for recordation and storage of the transformed data. In preferred embodiments, the user interface is used by the practitioner to manage various aspects of the machine, e.g., to direct the machine to carry out the various steps in the transformation of raw data into transformed data, recordation of the results of the transformation, and management of the transformed data stored in memory.

As such, in preferred embodiments, the methods further comprise a transformation of the computer-readable medium by recordation of the raw analytical reaction data and/or the transformed data generated by the methods. Further, the computer-readable medium may comprise software for providing a graphical representation of the raw analytical reaction data and/or the transformed data, and the graphical representation may be provided, e.g., in soft-copy (e.g., on an electronic display) and/or hard-copy (e.g., on a print-out) form.

The invention also provides a computer program product comprising a computer-readable medium having a computer-readable program code embodied therein, the computer readable program code adapted to implement one or more of the methods described herein, and optionally also providing storage for the results of the methods of the invention. In certain preferred embodiments, the computer program product comprises the computer-readable medium described above.

In another aspect, the invention provides data processing systems for transforming raw analytical reaction data from one or more analytical reactions into transformed data representative of a particular characteristic of an analytical reaction, e.g., an actual sequence of one or more template nucleic acids analyzed, a rate of an enzyme-mediated reaction, an identity of a kinase target molecule, and the like. Such data processing systems typically comprise a computer processor for processing the raw data according to the steps and methods described herein, and computer usable medium for storage of the raw data and/or the results of one or more steps of the transformation, such as the computer-readable medium described above.

### EXAMPLES

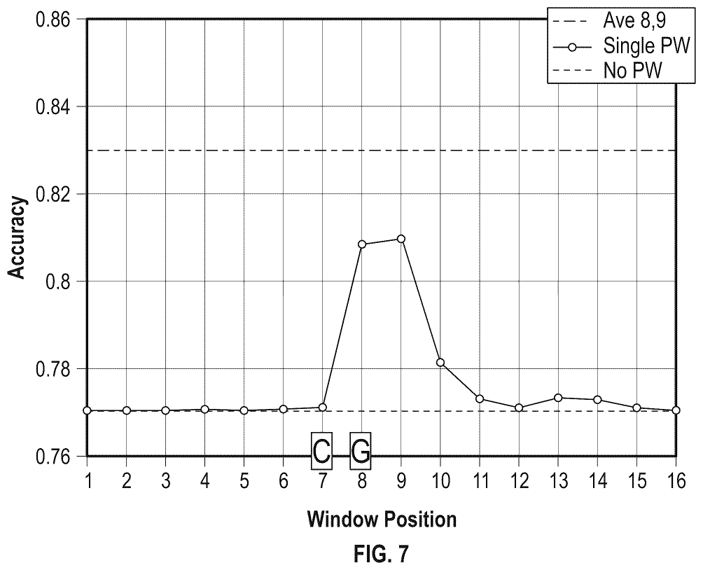

FIG. 7 shows the results from an experiment evaluating the performance of the methods of the invention. A series of models were run, and the accuracy of the methylation predictions were determined. Where no pulse width value was included, the accuracy was about 77%. When a single pulse width value was included, the accuracy increased to as much as about 81% when pulse width values for position 8 or 9 were used. When the method of the invention was used—here providing the average value of pulse width for positions 8 and 9 as a single value in position 8 of the feature vector, the accuracy of methylation prediction was 83%.

It is to be understood that the above description is intended to be illustrative and not restrictive. It readily should be apparent to one skilled in the art that various embodiments and—modifications may be made to the invention disclosed in this application without departing from the scope and spirit of the invention. The scope of the invention should, therefore, be determined not with reference to the above description, but should instead be determined with reference to the appended claims, along with the full scope of equivalents to which such claims are entitled. All publications mentioned herein are cited for the purpose of describing and disclosing reagents, methodologies and concepts that may be used in connection with the present invention. Nothing herein is to be construed as an admission that these references are prior art in relation to the inventions described herein. Throughout the disclosure various patents, patent applications, and publications are referenced. To the extent not already expressly incorporated herein, all published references and patent documents referred to in this disclosure are incorporated herein by reference in their entirety for all purposes.

What is claimed is:

1. A method for detecting 5-methylcytosine modifications in a nucleic acid template, the method comprising:
a) providing a nucleic acid template having a first strand and a complementary second strand, wherein the template is in a closed circular nucleic acid;
b) subjecting the circular nucleic acid template to a real-time single molecule sequencing process that incorporates fluorescently labeled nucleotides into a nascent strand by a polymerase enzyme, and measuring emitted signals to obtain a set of traces comprising pulses;
c) producing sequencing data by measuring features for each pulse in a trace of the real-time sequencing reaction, wherein said features comprise nucleotide identity, pulse width, and interpulse duration;
d) creating, from the sequencing data, a set of feature vectors, each feature vector comprising two nucleotide positions comprising a known CpG site, at least 3 nucleotide positions upstream of the CpG site and at least three nucleotide positions downstream of the CpG site, each feature vector comprising the input features: (i) nucleotide identity for each nucleotide position in the feature vector, (ii) interpulse duration for each nucleotide position in the feature vector, and (iii) average pulse width value for two or more nucleotides in the feature vector, and the average pulse width value provided at only a single position in the feature vector;
e) inputting the set of feature vectors into a model, the model trained using training feature vectors created from sequencing data obtained in the same manner and having the same input features recited in step (d), wherein one or more of the training feature vectors represent nucleic acids known to have 5-methylcytosine modifications, and one or more of the training feature vectors represent nucleic acids known to be free of 5-methylcytosine modifications; and
f) using the model to detect whether a cytosine (C) in the known CpG site of each feature vector has a 5-methylcystosine modification.

2. The method of claim 1, wherein the average pulse width value is the average pulse width value for two consecutive nucleotides.

3. The method of claim 1, wherein the feature vector comprises at least 5 nucleotide positions upstream and 5 nucleotide positions downstream of the known CpG site.

4. The method of claim 1, wherein the feature vector comprises 16 nucleotide positions.

5. The method of claim 1, wherein the feature vector has input features corresponding to the first strand.

6. The method of claim 1, wherein the feature vector has input features corresponding to both the first strand and the complementary second strand.

7. The method of claim 1, wherein a first feature vector with input features corresponding to the first strand and a second feature vector with input features corresponding to the second strand are input into the model to be processed by the model separately.

8. The method of claim 7, wherein the processed features from the first feature vector and second feature vector are combined.

9. The method of claim 8, wherein the processed features are combined using Bayesian inversion.

10. The method of claim 1, wherein the input features comprise consensus values obtained by combining multiple sequencing reads.

11. The method of claim 1, wherein the model comprises a neural network model.

12. The method of claim 11, wherein the neural network model comprises a convolutional neural network.

13. The method of claim 1, wherein the model comprises a deep learning model.

14. The method of claim 13, wherein the deep learning model comprises convolutional and pooling layers.

15. The method of claim 13, wherein the deep learning model comprises a full connection layer.

16. The method of claim 1, wherein the nucleic acid template is within a whole genome sequencing sample.

17. The method of claim 16, wherein 5-methylcytosine modifications are detected in a plurality of templates in the whole genome sequencing sample.

18. The method of claim 1, wherein the nucleic acid template comprises human DNA.

19. The method of claim 1, wherein the circular nucleic acid template comprises a fragment from between 10,000 and 15,000 bases connected at both ends by hairpin structures.

20. The method of claim 1, wherein the method is carried out on a sample comprising a plurality of nucleic acid templates.


SEQUENCE LISTING (3 sequences)

SEQ ID NO: 1 - DNA, 11 bp, synthetic construct
  source 1..11: mol_type = other DNA
agctagttcat

SEQ ID NO: 2 - DNA, 15 bp, synthetic construct
  source 1..15: mol_type = other DNA
gctcgatcaagtaca

SEQ ID NO: 3 - DNA, 27 bp, synthetic construct
  source 1..27: mol_type = other DNA
actgatatgtacgactgaggtctgagt